SEQUENCE LISTING (16 sequences)

SEQ ID NO: 1 - DNA, 1174 bp, Homo sapiens
atgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaagtttggggacatcccttgccggctggtgctcttcatgtttgccatgaaccgccagggcagcatcatattcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggcactgcaaatgtgtgcatcagcttcagcatctgccataccttccggtggcacgaagctatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccacatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaagctctagagc

SEQ ID NO: 2 - protein, 387 aa, Homo sapiens [sequence omitted]

SEQ ID NO: 3 - DNA, 20 bp, Homo sapiens
atgaatcggcaccatctgca

SEQ ID NO: 4 - DNA, 20 bp, Homo sapiens
tagagcttactcgatgcaac

SEQ ID NO: 5 - DNA, 20 bp, Homo sapiens
caaggtgttgccgccggtgt

SEQ ID NO: 6 - DNA, 20 bp, Homo sapiens
cagtgccattctggatcagc

SEQ ID NO: 7 - DNA, 24 bp, Artificial Sequence
  Description of Artificial Sequence adaptor sequence
agcactctccagcctctcaccgca

SEQ ID NO: 8 - DNA, 12 bp, Artificial Sequence
  Description of Artificial Sequence adaptor sequence
gatctgcggtga

SEQ ID NO: 9 - DNA, 24 bp, Artificial Sequence
  Description of Artificial Sequence adaptor sequence
accgacgtcgactatccatgaaca

SEQ ID NO: 10 - DNA, 12 bp, Artificial Sequence
  Description of Artificial Sequence adaptor sequence
gatctgttcatg

SEQ ID NO: 11 - DNA, 24 bp, Artificial Sequence
  Description of Artificial Sequence adaptor sequence
aggcaactgtgctatccgagggaa

SEQ ID NO: 12 - DNA, 12 bp, Artificial Sequence
  Description of Artificial Sequence adaptor sequence
gatcttccctcg

SEQ ID NO: 13 - DNA, 26 bp, Artificial Sequence
  Description of Artificial Sequence primer designed to amplify and clone  EX20 cDNA into expression vector
tcactagaattcatcatgaatcggca

SEQ ID NO: 14 - DNA, 24 bp, Artificial Sequence
  Description of Artificial Sequence primer designed to amplify and clone  EX 20 cDNA into expression vector
gtctagaagcttactcgatgcaac

SEQ ID NO: 15 - DNA, 1179 bp, Homo sapiens
atgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtagctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcgtctcttgccttctgtggggcatcactattggcctgacagtccacctcctgaagaagaagatgccgatccagaatggcggtgcaaatttgtgcagcagcttcagcatctgccataccttccagtggcacgaagctatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagataacaggtgagccagataataaccgcagcacgagcgtcgagctcacaggggaccccaacaaaaccagaggcgctccagaggcgttaatggccaactccggtgagccatggagcccctcttatctgggcccaacctctccttaaataaccatgccaagaagggacattgtcaccaagaaccaggatctctggagaaacagttgggctgttgcatcgagtaagctctagagc

SEQ ID NO: 16 - protein, 363 aa, Homo sapiens
MNRHHLQDHFLEIDKKNCCVFRDDFIVKVLPPVLGLEFIFGLLGNGLALWIFCFHLKSWKSSRIFLFNLAVADFLLIICLPFLMDNYVRRWDWKFGDIPCRLMLFMLAMNRQGSIIFLTVVAVDRYFRVVHPHHALNKISNRTAAIVSCLLWGITIGLTVHLLKKKMPIQNGGANLCSSFSICHTFQWHEAMFLLEFFLPLGIILFCSARIIWSLRQRQMDRHAKIKRAITFIMVVAIVFVICFLPSVVVRIRIFWLLHTSGTQNCEVYRSVDLAFFITLSFTYMNSMLDPVVYYFSSPSFPNFFSTLINRCLQRKITGEPDNNRSTSVELTGDPNKTRGAPEALMANSGEPWSPSYLGPTSP

(19) United States
(12) Patent Application Publication
(10) Pub. No.: US 2004/0038895 A1
(43) Pub. Date: Feb. 26, 2004

(54) Inflammation related G-protein coupled receptor

(76) Inventors: Gabor Jarai, Horsham (GB); Shida Yousefi, Bern (CH)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/619,141

(22) Filed: Jul. 14, 2003

Related U.S. Application Data
(63) Continuation of application No. 09/930,334, filed on Aug. 15, 2001, now abandoned.
(60) Provisional application No. 60/421,154, filed on Aug. 18, 2000.

(51) Int. Cl. A61K 38/17; A61K 48/00; C07H 21/04; C07K 14/705; C07K 16/28

(52) U.S. Cl. 514/12; 514/44; 536/23.1; 530/350; 530/388.1

(57) ABSTRACT

A pharmaceutical composition comprising as active ingredient (A) a polypeptide comprising the amino acid sequence of SEQ ID NO:2 or SEQ ID NO:16 or a functionally equivalent variant of the amino acid sequence; or (B) a polynucleotide comprising a nucleotide sequence encoding the polypeptide (A); or (C) an antibody which is immunoreactive with the polypeptide (A); or (D) an antisense oligonucleotide comprising a nucleotide sequence complementary to that of polynucleotide (B); and the use of (A), (B), (C) and (D) in diagnostic and therapeutic applications.

The present invention relates to the use of an inflammation-associated G-protein coupled receptor gene designated EX20, the protein molecule encoded by EX20 and related molecules in therapeutic and diagnostic applications.

Development and maintenance of several diseases, for example many respiratory and inflammatory diseases, involve the participation of a variety of cell types that undergo a number of phenotypic changes during the development of the pathological condition. These phenotypic alterations are the result of specific changes in the expression and functioning of various genes and proteins. The detection of genes or proteins, whose expression is altered in a particular physiological or pathological condition, can therefore lead to the identification of genes or proteins of pathological and therapeutic importance.

Cells that are attracted into tissues during inflammation include various inflammatory phagocytes such as neutrophilic and eosinophilic granulocytes and monocytes. These cells have been associated with inflammation and tissue destruction in several inflammatory diseases including respiratory tract inflammation in both acute and chronic bronchitis, chronic obstructive pulmonary disease (COPD), emphysema, asthma, adult respiratory distress syndrome (ARDS), rheumatoid arthritis, inflammatory bowel disease (IBD), ulcerative colitis, primary sclerosing cholangitis and Crohn's disease.

In several inflammatory respiratory diseases, there is an increased number of neutrophils, macrophages and other leukocytes present in the inflamed tissues. Enhanced migration into the lung, as a result of the release of chemoattractants by various leukocytes and epithelial cells and contribute to the accumulation of these cells at the sites of inflammation. In addition, increased levels of granulocyte macrophage colony stimulating factor (GM-CSF) have been shown to increase the functional life span of neutrophils and to increase both phagocytic and oxidative burst activity and the production of proinflammatory cytokines that are critical for regulating the inflammatory process in the abovementioned diseases.

Critical steps in the action of leukocytes in inflammatory conditions include the migration of these cells into the tissues, e.g. into the airways in respiratory inflammations or to the joints in rheumatoid arthritis, cell activation and the release of a range of inflammatory mediators, leukotrienes, oxygen radicals, proteases. The isolation of genes and proteins whose expression is upregulated upon cytokine stimulation, for example by GM-CSF, can identify molecular targets that can be exploited to offer therapeutic benefits.

Recently, several methods and technologies have been developed for the detection of differential gene expression and the isolation of differentially expressed genes. For example, changes can be identified at the protein level using proteomics approaches and changes in transcriptional regulation can be detected by several methods including differential display (Liang, P., and Pardee, A. B., Science 257:967-971), SAGE (serial analyses of gene expression) (Velculescu, .V. E., Zhang, L., Vogelstein, B., and Kinzler, K. W. Science, 270:484-487), differerential hybridization of complex cDNA probes high density cDNA or oligonucleotide arrays bound to solid support (Chee, M., Yang, R., Hubbell, E., Berno, A. Huang, X. C., Stern, D., Winkler, J., Lockhart, D. J., Morris, M.S. and Fodor, S. P. A. Science (1996) 274:610-614; Lockhart, D. J., Dong, H., Byrne, M. C., Follettie, M. T., Gallo, M. V., Chee, M. M., Wang, C., Kobayashi, M., Horton, H. and Brown, E. L. Nature Biotechnology (1996) 14:1675-1680; Shena, M., Shalon, D., Davis, R. W. and Brown, P.O. Science (1995) 250:467-470.) and cDNA subtraction methods such as representational difference analysis (Hubank, M., and Schatz, D. G. Nucleic Acids Res. 22: 5640-5648).

One method, which can be used to identify differentially expressed genes is Representational Difference Analysis of cDNA (cDNA-RDA). cDNA-RDA is a PCR-based subtractive enrichment procedure. Originally developed for the identification of differences between complex genomes it has been adapted to enable isolation of genes with altered expression between various tissues or cell samples (Lisitsyn, N., and Wigler, M. Science 259:946-951; Hubank, M., and Schatz, D. G. Nucleic Acids Res. 22: 5640-5648; O'Neill, M. J., and Sinclair, A. H. Nucleic Acids Res. 25: 2681-2682). This technique offers several advantages including the isolation of few false positives, the fact that unwanted difference products can be competitively eliminated and genes producing rare transcripts can also be detected and isolated.

Identification of genes that are over-expressed in inflammatory conditions would provide an important opportunity for the understanding of the inflammatory conditions. Tissue distribution and disease association of the over-expression of these genes and proteins can be established using tissue samples derived from appropriately selected patients. Various techniques, for example histology methods, such as in situ hybridisation and immunohistochemistry can be applied for this purpose. Over-expression of these genes would indicate their importance in the disease condition from which a number of clinically important applications would arise.

Identification of G-protein coupled receptors would be particularly advantageous. Signals that are needed for leukocyte migration and activation are often communicated through receptors that belong to the seven transmembrane-spanning G-protein coupled receptor (GPCR) superfamily characterised by seven transmembrane helices (TM-I through -VII) connected by three intracellular and three extracellular loops. The GPCR gene family is the largest known receptor family. GPCRs are transducers of extracellular signals and they allow tissues to respond to a wide array of signalling molecules. G-protein coupled receptors are important targets in therapeutic applications because they are involved in a wide variety of physiological and pathological processes. It is estimated that 60-70% of currently marketed drugs indeed act on members of the GPCR supetfamily.

Various cloning startegies and database mining approaches led to the cloning of a number of GPCRs. The identification of ligands, however, lags behind and there are a large number of GPCR genes whose protein products, using sequence similarity and predicted 3D structure as the criteria, are members of the GPCR family, but for which the ligands are not known. These receptors are commonly known as orphan G-protein coupled receptors (oGPCRs).

Identification of G-protein coupled receptor genes that are expressed in inflammatory cells and establishing the association of their over-expression with disease conditions would provide an important opportunity for the understanding of the inflammatory conditions from which a number of clinically important applications would arise. GPCRs identified may lead to the development of therapeutics (small molecule drugs, antisense molecules, antibody molecules) directly targeted to the gene or protein product of the gene, or may target the biochemical pathway at an upstream or downstream location if the development of such drugs is easier than directly targeting the gene. Polynucleotide sequences comprising the gene and sequence variants thereof may be used to develop a clinical diagnostic test for inflammatory conditions. Furthermore, information about the DNA sequences of GPCRs involved in inflammatory conditions and the amino acid sequences encoded by these genes facilitates large scale production of proteins by recombinant techniques and identification of the tissues and cells naturally producing the proteins. Such sequence information also permits the preparation of antibody substances or other novel binding molecules specifically reactive with the proteins encoded by the GPCR genes that may be used in modulating the natural ligand/antiligand binding reactions in which the proteins are involved.

It has been found that expression of EX20 is upregulated upon stimulation, for example by GM-CSF, of cytokines which are critical for regulating inflammatory processes in the abovementioned diseases. In view of the observed inflammatory disease-associated over-expression of EX20 in various leukocyte subsets, EX20 and the protein which it encodes are useful in the diagnosis of the aforementioned inflammatory diseases, and the encoded protein is useful as a therapeutic target for identification of agents suitable for treatment of those diseases.

Accordingly, the present invention provides, in one aspect, a pharmaceutical composition comprising as active ingredient (A) a polypeptide comprising the amino acid sequence of SEQ ID NO:2 or SEQ ID NO:16 or a functionally equivalent variant of said amino acid sequence, i.e. a variant thereof which retains the biological or other functional activity thereof, e.g. a variant which is capable of raising an antibody which binds to a polypeptide comprising the amino acid sequence of SEQ ID NO:2 or SEQ ID NO:16; or (B) a polynucleotide, hereinafter alternatively referred to as EX20, comprising a nucleotide sequence encoding the polypeptide (A); or (C) an antibody which is immunoreactive with the polypeptide (A); or (D) an antisense oligonucleotide comprising a nucleotide sequence complementary to that of polynucleotide (B); optionally together with a pharmaceutically acceptable carrier or diluent.

Terms used herein have the following meanings:

“Isolated” refers to material removed from its original environment.

“Hybridization” or “hybridizes” refers to any process by which a strand of a polynucleotide binds with a complementary strand through base pairing.

“Stringent conditions” refer to experimental conditions which allow up to 20% base pair mismatches, typically two 15 minute washes in 0.1×SSC (15 mM NaCl, 1.5 mM sodium citrate, pH 7.0) at 65° C.

“Homology” or “homologous” refers to a degree of similarity between nucleotide or amino acid sequences, which may be partial or, when sequences are identical, complete.

“Expression vector” refers to a linear or circular DNA molecule which comprises a segment encoding a polypeptide of interest operably linked to additional segments which provide for its transcription.

“Antisense” refers to selective inhibition of protein synthesis through hybridisation of an oligo- or polynucleotide to its complementary sequence in messenger RNA (mRNA) of the target protein. The antisense concept was first proposed by Zamecnik and Stephenson (Proc. Natl. Acad. Sci. USA 75:280-284; Proc. Natl. Acad. Sci. USA 75:285-288) and has subsequently found broad application both as an experimental tool and as a means of generating putative therapeutic molecules (Alama, A., Pharmacol. Res. 36:171-178; Dean, N. M., Biochem. Soc. Trans. 24:623-629; Bennet, C. F., J. Pharmacol. Exp. Ther. 280:988-1000; Crooke, S.T., Antisense Research and Applications, Springer).

The term “variant” as used herein means, in relation to amino acid sequences, an amino acid sequence that is altered by one or more amino acids. The changes may involve amino acid substitution, deletion or insertion. In relation to nucleotide sequences, the term “variant” as used herein means a nucleotide sequence that is altered by one or more nucleotides; the changes may involve nucleotide substitution, deletion or insertion. A preferred functionally equivalent variant of the amino acid sequence SEQ ID NO:2 or SEQ ID NO:16 is one having at least 80%, more preferably at least 90%, and especially more than 95% amino acid sequence identity to SEQ ID NO:2 or SEQ ID NO:16.

By an amino acid sequence having x % identity to a reference sequence such as SEQ ID NO:2 or SEQ ID NO:16, is meant a sequence which is identical to the reference sequence except that it may include up to 100 amino acid alterations per each 100 amino acids of the reference sequence. For example, in a subject amino acid sequence having at least 80% identity to a reference sequence, up to 20% of the amino acid residues in the reference sequence may be substituted, deleted or inserted with another amino acid residue. Percentage identity between amino acid sequences can be determined conventionally using known computer programs, for example the FASTDB program based on the algorithm of Brutlag et al (Comp. App. Biosci. (1990) 6:237-245).

The polynucleotide (B) may be cDNA, genomic DNA or RNA. In particular embodiments, the polynucleotide (B) is cDNA comprising the nucleotide sequence of SEQ ID NO:1 or SEQ ID NO:15 or a DNA comprising a nucleotide sequence which hybridises to SEQ ID NO:1 or SEQ ID NO:15 under stringent conditions. Nucleotide sequences which satisfy such hybridisation requirements include those resulting from deletions, insertions or substitutions of one or more nucleotides.

The invention also provides an isolated polynucleotide comprising the nucleotide sequence of SEQ ID NO:15.

In a further aspect of the invention, the polynucleotide (B) comprises a portion having at least 20, e.g. at least 50, e.g. at least 100, e.g. at least 200, e.g. at least 500, e.g. at least 1000, e.g. at least 1100 contiguous bases from SEQ ID NO:1 or SEQ ID NO:15. In a yet further aspect of the invention, the polynucleotide (B) comprises a nucleotide sequence encoding at least 10, e.g. at least 50, e.g. at least 100, e.g. at least 200, e.g. at least 300 contiguous amino acids from SEQ ID NO:2 or SEQ ID NO:16.

The polynucleotide (B) may be isolated by first isolating a fragment of it by PCR using degenerate primers that are designed using amino acid sequence motives conserved among members of a family or sub family of GPCRs. The degenerate primers can be used to amplify a fragment from cDNA that have been prepared from RNA isolated from human cells, specifically leukocytes or especially from phagocytes e.g neutrophilic and eosinophilic granulocytes or from genomic DNA. The isolated fragment is then sequenced and full lengths clones are obtained by first isolating overlapping fragments containing the 5′ and 3″ ends of the gene using 5′ and 3′ RACE (rapid amplification of cDNA ends) using gene specific primers designed using that sequence and RNA isolated from human cells, specifically from leukocytes or especially from phagocytes e.g neutrophilic and eosinophilic granulocytes or from genomic DNA and then joining those fragments together by standard methods.

The polynucleotide (B) may also be isolated by first isolating a fragment of it by PCR using degenerate primers that are designed using amino acid sequence motives conserved among members of a family or sub family of GPCRs. The degenerate primers can be used to amplify a fragment from cDNA that have been prepared from RNA isolated from human cells specifically from leukocytes and especially from phagocytes e.g neutrophilic and eosinophilic granulocytes or from genomic DNA. The isolated fragment is then sequenced and full lengths clones are obtained by using this fragment or a part thereof as probe for screening a human cDNA library, preferably a leukocyte or, especially, a granulocyte cDNA library or a human genomic DNA library.

The polynucleotide (B), for example having the sequence SEQ ID NO:1 or SEQ ID NO:15, may be prepared from the nucleotides which it comprises by chemical synthesis, e.g. automated solid phase synthesis using known procedures and apparatus.

The polypeptide (A) may be produced by cloning a polynucleotide sequence as hereinbefore described into an expression vector containing a promoter and other appropriate regulating elements for transcription, transferring into prokaryotic or eukaryotic host cells such as bacterial, plant, insect, yeast, animal or human cells, and culturing the host cells containing the recombinant expression vector under suitable conditions. Techniques for such recombinant expression of polypeptides are well known and are described, for example, in J. Sambrook et al, Molecular Cloning, second edition, Cold Spring Harbor Press, 1990.

In another aspect of the invention, the polypeptide (A) comprises a portion having at least 10, e.g. at least 50, e.g. at least 100, e.g. at least 200, e.g. at least 300 contiguous amino acids from SEQ ID NO:2 or SEQ ID NO:16.

The invention also provides an isolated polypeptide, particularly a recombinant polypeptide, comprising the amino acid sequence of SEQ ID NO:16.

The polypeptide (A) may be expressed as a recombinant fusion protein with one or more heterologous polypeptides, for example to facilitate purification. For example, it may be expressed as a recombinant fusion protein with a heterologous polypeptide such as a polyhistidine containing a cleavage site located between the polynucleotide sequence of the invention and the heterologous polypeptide sequence, so that the polypeptide comprising the amino acid sequence of SEQ ID NO:2 or SEQ ID NO:16 may be cleaved and purified away from the heterologous moiety using well known techniques.

The polypeptide (A) may also be synthesised, in whole or in part, from the amino acids which it comprises using well known chemical methods, for example automated solid phase techniques.

The polypeptide (A) may be purified by well known standard procedures.

The present invention also provides an antibody (C) which is immunoreactive with a polypeptide (A) as hereinbefore described. The antibody may be a polyclonal or monoclonal antibody. Such antibodies may be prepared using conventional procedures. Methods for the production of polyclonal antibodies against purified antigen are well established (cf. Cooper and Paterson in Current Protocols in Molecular Biology, Ausubel et al. Eds., John Wiley and Sons Inc., Chapter 11). Typically, a host animal, such as a rabbit, or a mouse, is immunised with a purified polypeptide (A), or immunogenic portion thereof, as antigen and, following an appropriate time interval, the host serum is collected and tested for antibodies specific against the polypeptide. Methods for the production of monoclonal antibodies against purified antigen are well established (cf. Chapter 11, Current Protocols in Molecular Biology, Ausubel et al. Eds., John Wiley and Sons Inc.). For the production of a polyclonal antibody, the serum can be treated with saturated ammonium sulphate or DEAE Sephadex. For the production of a monoclonal antibody, the spleen or lymphocytes of the immunised animal are removed and immortalised or used to produce hybridomas by known methods. Antibodies secreted by the immortalised cells are screened to determine the clones which secrete antibodies of the desired specificity, for example using Western blot analysis. Humanised antibodies can be prepared by conventional procedures.

In another aspect, the present invention provides an antisense oligonucleotide (D) comprising a nucleotide sequence complementary to that of a polynucleotide (B), in particular a nucleotide sequence complementary to SEQ ID NO:1 or SEQ ID NO:15. The antisense oligonucleotide may be DNA, an analogue of DNA such as a phosphorothioate or methylphosphonate analogue of DNA, RNA, an analogue of RNA, or a peptide nucleic acid (PNA). The antisense oligonucleotides may be synthesised by conventional me ods, for example using automated solid phase techniques.

The present invention also provides a polynucleotide probe comprising at least 15 contiguous nucleotides of a polynucleotide (B) as hereinbefore described or a complement thereof. The probe may be cDNA, genomic DNA or RNA. It may be, for example, a synthetic oligonucleotide comprising 15 to 50 nucleotides, or may be a longer molecule comprising, for example, up to 1100, e.g. up to 1000, e.g. up to 500 contiguous nucleotides of (B). The probe can be labelled, e.g. with a fluorophore or a chemiluminescent or radioactive label, to provide a detectable signal.

The polynucleotide probe is capable of selectively hybridising under stringent conditions to a polynucleotide fragment having a sequence SEQ ID NO:1 or SEQ ID NO:15. The probe has a sequence such that under such hybridisation conditions it hybridizes only to its cognate sequence. DNA probes as described above are useful in a number of screening applications including Northern and Southern blot analyses, dot blot and slot blot analyses, and fluorescence in situ hybridisation (FISH).

The present invention also includes a pair of oligonucleotides having nucleotide sequences useful as primers for DNA amplification of a fragment of a polynucleotide (B), i.e. of EX20, wherein each primer of said pair is at least 15 nucleotides in length and said pair have sequences such that when used in a polymerase chain reaction (PCR) with either human genomic DNA or a suitable human cDNA target they result in synthesis of a DNA fragment containing all or preferably part of the sequence of EX20. The primer pair is preferably capable of amplifying the coding region of EX20 or portion thereof. Examples of such primer pairs are shown hereinafter as SEQ ID NOs 3-4 and SEQ ID NOs 5-6 respectively.

The role of the polypeptide (A) in inflammatory diseases characterised by neutrophilic or eosinophilic inflammation can be determined using conventional allergen driven animal models for inflammatory conditions, e.g. an ovalbumin-induced mouse or rat model.

Polynucleotides, polypeptides, antibodies, antisense oligonucleotides or probes as hereinbefore described, hereinafter alternatively referred to collectively as agents of the invention, may be used in the treatment (prophylactic or symptomatic) or diagnosis of inflammatory diseases such as those hereinbefore described. In some inflammatory conditions, upregulation of EX20 may induce anti-inflammatory events, so that treatment which enhances this upregulation may be appropriate. For example, a polypeptide (A) may be used to treat a mammal, particularly a human, deficient in or otherwise in need of that polypeptide; a polynucleotide (B) may be used in gene therapy where it is desired to increase EX20 activity, for instance where a subject has a mutated or missing EX20 gene; an antibody (C) or an antisense oligonucleotide (D) may be used to inhibit EX20 activity, where this is desired; an antibody (C) may be used to detect, or determine the level of expression of, a polypeptide (A) or to inhibit ligand/antiligand binding activities of a polypeptide (A); and a probe of the invention may be used to detect the presence or absence of the EX20 gene, i.e. to detect genetic abnormality, or to determine the level of expression of EX20 in a cell sample, e.g. in diagnosis of inflammatory disease.

“Gene therapy refers to an approach to the treatment of human disease based upon the transfer of genetic material into somatic cells of an individual. Gene transfer can be achieved directly in vivo by administartion of gene-bearing viral or non-viral vectors into blood or tissues, or indirectly ex vivo through the introduction of genetic material into cells manipulated in the laboratory followed by delivery of the gene-containing cells back to the individual. By altering the genetic material within a cell, gene therapy may correct underlying disease pathophysiology. Suitable vectors, and procedures, for gene delivery to specific tissues and organ systems in animals are described in Dracopoli, N. C. et al., Current Protocols in Human Genetics. John Wiley and Sons Inc., Chapters 12 and 13 respectively. In relation to a polynucleotide (A), gene therapy may involve delivery of a viral or non-viral gene therapy vector containing an expression cassette of the EX20 gene under suitable control elements to the lungs of diseased individuals (eg. asthmatics) so that the underlying disease pathophysiology is corrected or ameliorated.

Accordingly, in further-aspects,-the-present invention provides:

a method of treating an inflammatory disease, particularly an inflammatory or obstructive airways disease, which comprises administering to a subject in need thereof an effective amount of a polypeptide (A), a polynucleotide (B), an antibody (C) or an” antisense oligonucleotide (D) as hereinbefore described;

use of a polypeptide (A), a polynucleotide (B), an antibody (C) or an antisense oligonucleotide (D) as hereinbefore described for the preparation of a medicament for the treatment of an inflammatory disease;

a method of detecting genetic abnormality or a predisposition to disease in a subject which comprises-incubating a genetic sample from the subject with a polynucleotide probe of the invention as hereinbefore defined, under conditions where the probe hybridises to complementary polynucleotide sequence, to produce a first reaction product, and comparing the first reaction product to a control reaction product obtained with a normal genetic sample, where a difference between the first reaction product and the control reaction product indicates a genetic abnormality in the subject or a predisposition to disease;

a method of detecting the presence of a polynucleotide (B), e.g. comprising SEQ ID NO:1 or SEQ ID NO: 15, in cells or tissues which comprises contacting DNA from the cell or tissue with a polynucleotide probe as hereinbefore defined under conditions where the probe is specifically hybridizable with a polynucleotide (B), and detecting-whether hybridization occurs; and

a method of detecting an abnormality in the nucleotide sequence of a polynucleotide (B) in a patient which comprises amplifying a target nucleotide sequence in DNA isolated from the patient by a polymerase chain reaction using a pair of primers as hereinbefore described which target the sequence to be amplified and analysing the amplified sequence to determine any polymorphism present therein.

The term “polymorphism” means any sequence difference as compared with the sequence of a polynucleotide (B) as hereinbefore described.

Hybridisation of a polynucleotide probe of the invention with complementary polynucleotide sequence may be detected using in situ (eg. FISH) hybridization, Northern or Southern blot analyses, dot blot or slot blot analyses. The abnormality may also be detected for example by conformation sensitive gel electrophoresis (CSGE) and DNA sequencing. The genetic abnormality may result in a change in the amino acid sequence of the individual's EX20 protein relative to the the amino acid sequence of a normal EX20 protein, or loss of protein. Alternatively, the change may not alter the amino acid sequence but may instead alter expression of the EX20 gene by altering the sequence of controlling elements either at the 5′-, or 3′-end of the gene, or altering the sequence of control elements within intronic regions of the gene. Changes may also affect the way the gene transcript is processed or translated. The invention also includes kits for the detection of an abnormality in the polynucleotide sequence of an individual's EX20 gene or for determining the level of expression of EX20 in an individual's cells. Hybridisation kits for such use comprise a probe of the invention as hereinbefore described, which probe may be modified by incorporation of a detectable, e.g. chemiluminescent, radioactive or fluorescent, label therein, and may include other reagents such as labelling reagents, i.e. reagents to incorporate a detectable label such as a radioactive isotope, chemiluminescent or fluorescent group into a hybridised product, and buffers. PCR amplification kits comprise primer pairs such as those described above together with a DNA polymerase such as Taq polymerase, and may include additional reagents, such as an amplification buffer and the like. Specific embodiments of the PCR amplification kits can include additional reagents specific for a number of techniques that detect polynucleotide changes,: including CSGE and DNA sequencing.

Determination of the level of expression of a polynucleotide (B) can be used in diagnosis of inflammatory diseases such as those abovementioned. Accordingly, the invention includes a method of determining whether a subject has an inflammatory disease, for example an inflammatory disease associated with increased GM-CSF levels, comprising determining, in a cell sample from the subject, the level of expression of a polynucleotide (B) as hereinbefore described, particularly a polynucleotide (B) comprising the nucleotide sequence of SEQ ID NO:1 or SEQ ID NO:15, or a nucleotide sequence which hybridises thereto under stringent conditions, and comparing said level with the level of expression of the polynucleotide in a cell sample from a healthy subject. An increased level of expression of a polynucleotide (B) indicates an inflammatory disease. The measured level indicates the nature of the inflammatory disease. The level of expression of a polynucleotide (B) may be determined, for example, by Nothern blot analysis, reverse transcription-polymerase chain reaction (RT-PCR), in situ hybridization, immunoprecipitation, Western blot hybridization or immunohistochemistry.

For example, in a diagnostic test using quantitative RT-PCR, mRNA is isolated from cells of interest, for example from BAL fluid cells or peripheral blood cells. Two primers are designed, one with identical sequence to the region between nucleotides 765 and 783 of SEQ ID NO: 1, the other with a nucleotide sequence that is the reverse complement of nucleotides 815-835 of SEQ ID NO: 1. An appropriately modified and labelled probe is then made corresponding to the reverse complement of the sequence between nucleotides 787-813 of SEQ ID NO: 1. Using appropriate control genes, these two primers and probe are used in TaqMan (PE Applied Biosystems) equipment to determine the mRNA quantity of EX20 in samples obtained from patients under investigation and healthy subjects. When in situ hybridisation is used for diagnostic purposes, a probe of approximately 200 to 1200 bp corresponding to any part of the sequence shown in SEQ ID NO: 1 can be used. Several overlapping probes of different length can be used in such a test, for example the one described in Example 3. Samples can be collected from patients and embedded in paraffin. Such samples can be, for example, peripheral blood cells, bronchoalveolar lavage (BAL) fluid cells, or various tissue sections. Labelled riboprobes prepared as described in Example 3 can then be used to determine the expression level of EX20 in those samples and thereby identify the inflammatory condition.

The invention also includes a method of monitoring treatment of a subject having an inflammatory disease such as hereinbefore described, e.g. such a disease associated with increased GM-CSF levels, with a drug such as those hereinbefore described as agents of the invention, which comprises determining the level of expression of a polynucleotide (B) or a polypeptide (A) as hereinbefore described or the level of an activity of said polypeptide in a cell sample from the subject following the treatment and comparing said level with the respective level before the treatment. Where the comparison indicates it to be desirable, administration of the drug to the subject may be altered accordingly.

The effectiveness of an agent of the invention in inhibiting or reversing inflammatory conditions, for example in inflammatory airways diseases, may be demonstrated in an animal model, e.g. a mouse or rat model, of airways inflammation or other inflammatory conditions, for example as described by Szarka et al, J. Immunol. Methods (1997) 202:49-57; Renzi et al, Am. Rev. Respir. Dis. (1993) 148:932-939; Tsuyuki et al., J. Clin. Invest. (1995) 96:2924-2931; Cernadas et al (1999) Am. J. Respir. Cell Mol. Biol. 20:1-8; Durie et al., Clin. Immunol. Immunopathol.(1994) 73: 11-18; and Williams et al., Proc. Natl. Acad. Sci. USA (1992) 89:9784-9788.

Inflammatory diseases to which the present invention is applicable include inflammatory or obstructive airways diseases such as asthma of whatever type or genesis, including both intrinsic (non-allergic) asthma and extrinsic (allergic) asthma. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Other inflammatory or obstructive airways diseases and conditions to which the present invention is applicable include adult respiratory distress syndrome (ARDS), chronic obstructive pulmonary or airways disease (COPD or COAD), including chronic bronchitis, or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Having regard to their anti-inflammatory activity, in particular in relation to inhibition of neutrophil or eosinophil activation, agents of the invention are also useful in the treatment of neutrophil or eosinophil related disorders, e.g. neutrophilia or eosinophilia, in particular neutrophil or eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction; and neutrophil-related disorders such as acute and chronic bronchitis, COPD, ARDS, emphysema, rheumatoid arthritis, inflammatory bowel disease (IBD), ulcerative colitis, primary sclerosing cholangitis and Crohn's disease. Agents of the invention are also useful in the treatment of inflammatory skin diseases such as eczematous dermatitis.

The agents of the invention may be administered by any appropriate route, e.g. orally, for example in the form of a tablet or capsule; parenterally, for example intravenously; topically, e.g. in an ointment or cream; transdermally, e.g. in a patch; by inhalation; or intranasally.

Pharmaceutical compositions containing agents of the invention may be prepared using conventional diluents or excipients and techniques known in the galenic art. Thus oral dosage forms may include tablets and capsules, and compositions for inhalation may comprise aerosol or other atomizable formulations or dry powder formulations.

The invention includes (i) an agent (A), (B), (C) or (D) of the invention in inhalable form, e.g. in an aerosol or other atomizable composition or in inhalable particulate, e.g. micronised form, (ii) an inhalable medicament comprising an agent (A), (B), (C) or (D) of the invention in inhalable form; (iii) a pharmaceutical product comprising an agent (A), (B), (C) or (D) of the invention in inhalable form in association with an inhalation device; and (iv) an inhalation device containing an agent (A), (B), (C) or (D) of the invention in inhalable form.

Dosages of agents of the invention employed in practising the present invention may of course vary depending, for example, on the particular condition to be treated, the effect desired and the mode of administration. In general, suitable daily dosages for administration by inhalation are of the order of 1 μg to 10 mg/kg while for oral administration suitable daily doses are of the order of 0.1 mg to 1000 mg/kg.

A polypeptide (A) as hereinbefore described can be used to identify enhancers (agonists) or inhibitors (antagonists) of its activity, i.e. to identify compounds useful in the treatment of inflammatory diseases, particularly inflammatory or obstructive airways diseases. Accordingly, the invention also provides a method of identifying a substance suitable for use in the treatment of inflammatory diseases comprising combining a candidate substance with a polypeptide (A) as hereinbefore described and measuring the effect of the candidate substance on the activity of said polypeptide (A). The activity of the polypeptide (A) may be measured, for example, by measuring intracellular Ca²⁺ or cAMP (cyclic AMP) levels or by a change in shape or by an appropriate reporter gene assay. The invention also includes a method of identifying a substance suitable for use in the treatment of inflammatory diseases which binds to a polypeptide (A) as hereinbefore described comprising mixing a candidate substance with said polypeptide (A) and determining whether binding has occurred.

The invention is illustrated by the following Examples. Abbreviations used in the Examples have the following meanings:

| | |
|---|---|
| BLAST: | basic local alignment search tool |
| BSA: | bovine serum albumin |
| cAMP: | cyclic adenosine monophosphate |
| DTT: | dithiothreitol |
| EDTA: | ethilene-diamine tetra acetic acid |
| EIA: | enzyme immunoassay |
| EST: | expressed sequence tag |
| FCS: | fetal calf serum |
| GM-CSF: | granulocyte macrophage colony stimulating factor |
| HEPES: | 4-(2-hydroxyethyl)piperazine-1-ethanesulfonic acid |
| IPTG: | isopropyl-b-D-thiogalactopiranoside |
| LMP: | low melting point |
| MOI: | multiplicity of infection |
| PBS | phosphate buffered saline |
| PEG: | polyethylene glycol |
| PBMC: | peripheral blood mononuclear cells |
| PCR: | polymerase chain reaction |
| PMSF: | phenylmethylsulfonyl fluoride |
| RPMI: | Rosewell Park Memorial Institute |
| SDS-PAGE: | sodium dodecyl sulfate polyacrylamide gel electrophoresis |
| TEV: | tobacco etch virus |

### EXAMPLE 1

Blood (200 ml) is collected in tubes containing sodium citrate under sterile conditions from normal donors with no history of respiratory diseases. Neutrophils are purified by well established methods. PBMC are separated from peripheral blood cells by Ficoll Hypaque (Pharmacia) centrifugation. The remaining cell population, mainly granulocytes and erythrocytes, are treated with erythrocyte lysis solution (155 mM NH₄Cl, 10 mM KHCO₃, 0.1 mM EDTA, PH 7.3). To determine the purity, granulocytes are stained with Hansel stain (Difco Laboratories Ltd) and are differentiated by light microscopy at high power magnification. The contamination with eosinophils is found to be less than 2%. For stimulation, neutrophils are resuspended at a concentration of 5 million cells per ml in RPMI-1640 plus 10% FCS. Cells are cultured for 5 hours with or without 50 ng/ml human recombinant GM-CSF (R&D System). Total RNA is extracted using TRIZOL Reagent (Gibco/BRL) as described by the manufacturer. One ml of TRIZOL is used for resuspension of every 5 million pelleted neutrophils. mRNA is purified using the MESSAGEMAKER mRNA isolation kit (Gibco/BRL) using conditions recommended by the manufacturer. 300 ng of mRNA is used to synthesize cDNA using the Superscript Choice System (Gibco/BRL) and oligo(dT) primer for first strand synthesis. Double stranded cDNA is extracted once with Approximately 10 ng of cDNA is ligated to 25 ng pCR2.1 vector (Invitrogen) and the ligation introduced into 50 μl One Shot competent cells (Invitrogen). The libraries are plated onto agar plates containing 50 μg/ml carbenicillin, and 100 mM IPTG and 50 μg/ml X-Gal. Plates are incubated at 37° C. overnight and then briefly at 4° C. to allow blue/white staining to be clearly distinguishable and plasmids are purified from 3 ml cultures of the white-colonies. Inserts of individual clones are analysed by determining their nucleotide sequence on an automated ABI310 sequencer (Perkin-Elmer) using M13 reverse and forward primers. The resulting sequences are analysed in sequence similarity searches using the BLAST algorithm and sequence alignments are done using the GCG software package (Wisconsin Package Version 9.1). A clone called EX20 is identified as containing an insert with significant sequence similarity to the database entry of the human orphan GPCR HM74 (GenBank accession number D10923;Nomura et al.: Molecular cloning of cDNAs encoding a LD78 receptor and putative leukocyte chemotactic peptide receptors. Int. Immunol. S(10):1239-1249, 1993). The full length coding region of the EX20 gene is then isolated by PCR using the known sequence in the public database. Amplification products are analysed by determining their nucleotide sequences on both strands on an automated ABI310 sequencer (Perkin-Elmer) using M13 reverse and forward primers as well as gene specific primers. The resulting sequences are analysed and sequence contig is obtained using ,the GCG software package-(Wisconsin-Package-Version 9.1). The obtained cDNA sequence contig (SEQ ID NO:1) shows 99% sequence identity with HM74 in the studied region. Translation of this nucleotide sequence results in a protein of 387 amino acid residues as shown in SEQ ID NO:2. Other sequence variants of the EX20 gene are also isolated. One of these variants shown in SEQ ID NO:15 is characterised by several single nucleotide differences along the coding region and a 5 bp-long deletion between nucleotide positions 1086-1090 of SEQ ID NO:1. Translation of this nucleotide sequence results in an open reading frame of 363 amino acids as shown in SEQ ID NO:16. This version of the EX20 protein is characterised by 18 single amino acid substitutions along the protein sequence and a truncation resulting in a protein that has an intracellular C-terminus which is shorter by 24 amino acid residues when compared to the protein sequence shown in SEQ ID NO:2. These amino acid sequence differences may have functional consequences for example in ligand binding, receptor activation or downstream signalling events coupled to the EX20 receptor.

### EXAMPLE 3

This example describes the analysis of the expression and tissue distribution of the EX20 gene by in situ hybridisation.

For the generation of labeled riboprobes a subclone is constructed containing an 1161 bp insert between the EcoRI and XbaI sites of the pSPORT1 plasmid (Life Technologies). The insert of this subclone is identical to the insert described below in Example 4 and is generated in an identical manner. In this construct using the SP6 promoter in the vector transcribes the antisense strand of the insert that can be used as probe in the in situ hybridisation experiments. The T7 promoter in the vector is used to transcribe the sense strand, which is used as a negative control. Serial tissue sections from paraffin-embedded samples are hybridized with radiolabeled cRNA probes that are synthesized from the 1.2 kb insert. Riboprobes are transcribed in vitro in the presence of ³³P-uridine 5′-triphosphate with SP6 (antisense) and T7 (sense) RNA polymerases. The probes are then column-purified and then subjected to electrophoresis on a 5% TBE-urea acrylamide gel to confirm size and purity. Tissue sections are digested with Proteinase K and then hybridized with the probes at approximately 8.0×10⁸ dpm/ml at 65° C. for 18 hours. Slides are treated with RNAse A and washed stringently for 2 hours in 0.1×SSC at 65° C. The slides are then coated with Kodak NTB-2 emulsion, exposed for 7 days at 4° C., and developed using Kodak D-19 Developer and Fixer. Slides are stained with hematoxylin and eosin and imaged using a Sony Digital Photo Camera attached to a Nikon microscope. In addition to hybridization with the antisense probe, alternate sections are hybridized with two types of control probes. All tissues are initially screened with a probe for beta-actin mRNA to ensure that RNA has been preserved within the samples. Adjacent serial sections are also hybridized with a sense control riboprobe derived from the same region of the gene as the antisense probe.

The images are evaluated and it is found that the EX20 gene is preferentially overexpressed in tissues affected by various inflammatory diseases in leukocyte subsets. Strong signals are observed in macrophages and neutrophils in respiratory inflammations in COPD, emphysema, ARDS and asthma, in synovial histio-monocytes in rheumatoid arthritis, in neutrophils and epithelioid histiocytes in Crohn's disease. A subset of lyphocytes also shows intensive signals in the airways in asthma and COPD, in the synovium in rheumatoid arthritis and in the inflammatory infiltrates in eczematous dermatitis. The intensity of the signals in the inflammatory cells is consistently greater in inflamed tissues compared to the intensity of signal in these same cell types in non-inflamed or normal appearing tissues.

### EXAMPLE 4

This example relates to the expression of the full length functional EX20 in a mammalian expression system using stable transfection and the use of the transfected cells for the identification of natural ligands or artificial agonists of the EX20 protein.

Construction of the Expression-Vector

A unique EcoRI site (GAATTC) is incorporated 5′ to the EX20 start codon (ATG) by PCR amplification using the following primer (SEQ ID NO:13):

5′-TCACTAGAATTCATCATGAATCGGCA-3′

Another primer (SEQ ID NO: 14) is used to introduce a unique XbaI (TCTAGA) site 3′ to the EX20 stop codon (TAA, reverse complement: TTA);

5′- GTCTAGAAGCTTACTCGATGCAAC-3′

The recombinant amplified product is digested with EcoRi and XbaI restricion enzymes and ligated as a 1176 bp fragment into EcoRI/Xbal digested pcDNA3.1(+) (Invitrogen) mammalian expression vector and transformed into E. coli DHS™ cells. Transformants are selected using the ampicillin resistance gene present on pcDNA3.1(+) and recombinant vectors containing the EX20 insert are identified by isolating plasmids from randomly selected colonies and analysing the plasmids by restriction digestion and agarose gel electrophoresis using standard methods.

Stable Expression of EX20 in Mammalian Cells

The recombinant EX20 insert containing plasmid vector is then transfected into CHO-K1 cells. A confluent flask of CHO cells grown in Dulbecco's Modified Eagle's Medium/Ham's fl2 (50:50) with 10% FCS and 2 mM glutamine is trypsinized and plated at a dilution of 1:20 into 2 wells of a 6-well plate in 2 ml/well of the same medium. Cells are then incubated for 24 hours at 37° C. with 5% CO₂. The next day the transfection mix is prepared. 1 μg plasmid DNA is mixed into 100 μl OptiMEM serum free medium (Life Technologies) for each well. For each well 100 μl Lipofectamine is diluted in 100 μl OptiMEM in a separate tube. The two solutions are mixed and incubated for 15 minutes at room temperature. During incubation, cells are washed with OptiMEM to remove serum. 0.8 ml/transfection of serum-free OptiMEM is then added to the DNA-liposome transfection mix and 1 ml of that solution is then added to each well. Control cells are treated in an identical manner but omitting plasmid DNA from the transfection mix. Cells are then incubated for 5 hours at 37° C. with 5% CO₂ and then the transfection mix is replaced with 2 ml normal growth medium. After 24 hours transfectants are selected by washing cells in PBS, trypsinizing and re-plating them into T75 flasks with 1 mg/ml G418 (Life Technologies). Cells are then incubated at 37° C. with 5% CO₂, regularly changing the medium every 2 days until cells in the control flask have died. Cells are then dilution cloned by placing them at-a-density of one cell-per well into individual wells of a 96-well plate and growing them to confluence. After further expansion of the cells individual colonies are -screened for the expresssion of EX20 by RT-PCR and by using poly- or monoclonal antibodies raised against the EX20 protein.

Identification of Natural Ligands and Agonists Using Intracellular Calcium Assay

The transfectant cell line stably expressing the EX20 receptor protein and non transfected controls are grown to confluence in T162 flasks, trypsinized and resuspeneded in an appropriate volume, approximately 50 ml of growth medium with no antibiotic. Cells are seeded at 30,000 cells/well in 100 μl/well into 96-well plates that will allow the formation of confluent mono-layers at the time of assay the next day. After 24 hours cells are incubated with cytoplasmic calcium indicator Fluo-3-AM (4 mM) in 100 ml cell culture medium containing 20 mM HEPES and 2.5 mM probenecid at 37° C. for 60 min. Cells are washed 4 times with PBS containing 20 mm HEPES and 2.5 mM probenecid and 100 ml of that solution is then added to each well. The test compounds from collections of natural ligands and synthetic compound libraries are added to the cells and the fluorescent signal is read every second for the first 60 seconds and every 5 seconds for the next 30 seconds. Natural or synthetic agonists are identified by comparing the level of signal generated by the same compound in EX20 expressing and non-expressing control cells.

### EXAMPLE 5

This Example relates to the expression of full length EX20 with a 6 histidine tag after the ATG start codon using the Baculovirus system in Spodoptera frugiperda Sf9 cells, and to the purification of the resulting polypeptide.

Construction of a Recombinant EX20 Baculovirus

A unique EcoRI site (GAATTC) is incorporated 5′ to the EX20 start codon (ATG) by PCR amplification using the following primer (SEQ ID NO:13)

5′- TCACTAGAATTCATCATGAATCGGCA-3′

Another primer (SEQ ID NO: 14) is used to introduce a unique XbaI (TCTAGA) site 3′ to the EX20 stop codon (TAA, reverse complement: TTA).

5′-GTCTAGAAGCTTACTCGATGCAAC-3′

The recombinant amplified product is digested with EcoRI and XbaI restricion enzymes and ligated as a 1176 bp fragment into EcoRI/XbaI digested pFastbac™HTa baculovirus transfer vector (Life Technologies). In this construct the EX20 gene is expressed as a fusion potein as the EX20 coding region is placed after a 6×His affinity tag followed by a spacer region, a recognition site for TEV protease and an additional 7 amino acid linker region. Expression of the EX20 fusion protein containing the 6×His tag aids affinity purification and the TEV protease cleavage site is used to remove the 6×His tag. The recombinant EX20 sequence is transposed into Bacmid DNA carried by DHlOBac cells (Life Technologies; Bac to Bac Baculovirus expression system). EX20 recombinant Bacmids are isolated from DHlOBac cells and successful transposition is confirmed by PCR analyses.

Transfection of Sf9 Cells with Recombinant EX20 Bacmid DNA and Amplification of Recombinant Baculovirus Stocks

Recombinant EX20 Bacmid DNA is transfected into Sf9 cells using published protocols (Bac to Bac baculovirus expression system manual; Life Technologies). Recombinant baculoviruses are harvested from the culture medium after 3-day incubation at 27° C. The cell supernatants are clarified by centrifugation for 5 minutes at 500×g and kept at 4° C. The recombinant Baculovirus is amplified by infecting Sf9 cells (SF900 SFMII medium; Life Technologies) at a cell density of 1×10⁶ cells/ml and a multiplicity of infection (MOI) of 0.01 for 48 hours. Sf9 cells are then centrifuged at 1000×g for 5 minutes. The supernatants containing high titre virus are stored at 4° C.

Expression of Recombinant EX20 in Sf9 Cells

Sf9 cells, maintained at densities of between 2×10⁵ and 3×10⁶ cells/ml in SF00 SFMII medium; Life Technologies) in either shaker flasks (rotated at 90 RPM) or spinner flasks (stirring at 75 RPM) are infected with the amplified recombinant Baculovirus at a cell density of 1.5×10⁶ at an MOI of 2.0 for 60 hours. Following infection Sf9 cells are centrifuged at 1000×g for 5 minutes, the supernatants poured off and the cell pellets frozen at −80° C.

Crude Lysate Preparation

The cells (1×10⁹) are resuspended in 100 ml lysis buffer (20 mM Hepes pH 7.9, 100 mM NaCl, 5% glycerol, 2 mM E-mercaptoethanol, 0.5 mM imidazole, 0.1% Nonidet P-40, 40 pg/ml AEBSF, 0.5 pg/ml leupeptin, 1 pg/ml aprotinin and 0.7 pg/ml pepstatin A). Cells are incubated on ice for 15 minutes then centrifuged at 39,000×g for 30 minutes at 4° C.

Metal Chelate Affinity Chromatography

Metal chelate affinity chromatography is carried out at room temperature with a column attached to a BioCAD chromatography workstation. A 20 ml Poros MC/M (16 mmD×100 mmL) column is charged with Ni²⁺ prior to use and after each injection. To charge with Ni²⁺, the column is washed with 10 column volumes (CV) 50 mM EDTA pH 8, 1 M NaCl followed by 10CV water. The column is charged with 500 ml 0.1 M NiSO4 pH 4.5-5, washed with 10CV water, then any unbound Ni²⁺ removed by washing with 5CV 0.3 M NaCl. All steps are performed with a flow rate of 20 ml/min. The charged MC/M column is equilibrated with 5CV Buffer B (20 mM Hepes pH 7.9, 100 mM NaCl, 5% glycerol, 2 mM E-mercaptoethanol, 1 mM PMSF, 100 mM imidazole) to saturate the sites followed by 10CV Buffer A (as Buffer B except 0.5 mM imidazole). 90-95 ml of the crude lysate is loaded onto the column per run at a flow rate of 20 Ml/min. Subsequent steps are carried out with a flow rate of 30 ml/min. Any unbound material is removed by washing with 12 CV buffer A and EX20 eluted by applying a 0-50% Buffer B gradient over 10 CV. Fractions (8 ml) are collected over the gradient. EX20 containing fractions are combined and protease inhibitors added to the final concentrations described for the lysis buffer above. DTT is also added to a final concentration of 1 mM. The combined fractions are dialysed overnight against 4 litres 20 mM Hepes pH 7.9, 1 mM DTT, 0.2 mM PMSF at 4° C. The protein concentartion is determined and, if needed, samples are concentrated using a Millipore Ultrafree-15 centrifugation device (MW cut-off 50 kDa) at 4° C. The device is pre-rinsed with water prior to use. The final storage buffer used for long term storage at −80° C. is 20 mM Hepes pH 7.9, 1 mM DTT, ˜100 mM NaCl, 5% glycerol. Glycerol can be omitted from the sample for storage at 4° C.

### EXAMPLE 6

This example relates to the generation of polyclonal antibodies against the EX20 protein.

Immunisation

Rabbits are immunised at 4 subcutaneous sites with 500 pg purified EX20 protein according to the following schedule:

| DAYS | IMMUNISATIONS |
|---|---|
| 0 | 1^{st} immunisation 1:1 in complete Freund's adjuvant |
| 15 | 1^{st} boost 1:1 in incomplete Freund's adjuvant |
| 45 | 2^{nd} boost 1:1 in incomplete Freund's adjuvant |
| 55 | 1^{st} test bleed from the ear artery |
| Every | Boost 1:1 in incomplete Freund's adjuvant until a good antibody |
| month | response is obtained |

Test bleeds (500 μl) are taken and the serum assessed for antibody titre. Serum is collected when a maximum titre is reached. This is done by collecting blood (10 ml) and allowing it to clot for 2 hours at 4° C. The blood is centrifuged at 1000×g for 5 minutes to separate the serum. The serum is removed and stored at −20° C. until assayed.

ELISA Screening

Nunc-Immuno Plate Maxisorp 96 well plates (Nunc, Basle, CH) are used as a solid support and coated with the purified EX20 protein (100 ng/well) overnight at 4° C. The plates are blocked for 3 hours at 37° C. with PBS containing 2% BSA (Sigma) and 0.02% NaN₃ (Sigma). After blocking, plates are incubated overnight at room temperature with plasma in different dilutions of PBS. The presence of polyclonal antibodies is checked with both biotin labelled IgG-antibodies to rabbit (Goat anti-rabbit IgG antiserum, 1:25000 dilution), with an incubation time of 40 minutes. Alkaline phosphatase conjugated streptavidin (Immununo Research, Dianova, CH) is then added at a dilution of 1:10000. Development of the reaction is carried out by adding phosphate substrate (Sigma, f.c. 1 mg/ml) dissolved in diethanolamine. After 45 minutes, absorbance is read at 405 nm with a reference of 490 nm with an ELISA plate reader (Biorad).

Purification of the Polyclonal Antibodies

5 ml protein A-agarose is poured into a chromatography column and washed with 6 column volumes of 0.1 M tris (hydroxymethyl) methylamine (Tris) buffer pH 7.5. The rabbit serum containing anti-EX20 antibodies is diluted (½) with Tris buffer and added to the protein A-agarose. Unbound proteins are removed by washing the column with 6 volumes of Tris buffer. The IgG is eluted off the column with three column volumes of 0.1 M glycine buffer pH 3.0 and collected as 1 ml fractions into tubes containing 28 μl of 1 M Tris. The fractions which are positive for protein content are checked for purity by SDS-PAGE under reducing conditions. Two bands at 50 and 25 Kd are visualised corresponding to the heavy and light chains of an immunoglobulin molecule. Fractions containing only immunoglobulin are pooled, rechecked for protein concentration and stored at −20° C.

### EXAMPLE 7

This example relates to the generation of monoclonal antibodies against the EX20 protein.

Immunisation

Female Balb/c mice are immunised intraperitoneally (ip) with 100 μg of EX20 protein according to the schedule given below:

| DAYS | IMMUNISATIONS |
|---|---|
| 1 | 1^{st} immunisation 1:1 in complete Freund's adjuvant |
| 14 | 1^{st} boost 1:1 in incomplete Freund's adjuvant |
| 21 | 2^{nd} boost 1:1 in incomplete Freund's adjuvant |
| 28-30 | Three final boosts in PBS |
| 31 | Fusion with mouse myeloma cells |

Serum is assessed for antibody titre by ELISA (Example 6) after the animal is sacrificed for the preparation of spleen cells for fusion. If antibody titre is sufficient, (1/1000 to 1/100,000), the hybridomas are screened, otherwise discarded.

Preparation of Myeloma Cells

Sp2/0 murine myeloma cells (ATCC #CRL 1581; maintained in culture medium containing 20 μg/ml 8-azaguanine) are cultivated for one week before fusion in RPMI 1640 (8-azaguanine is not included), 10% (v/v) FCS and 1% penicillin-streptomycin (50 IU/ml and 50 μg/ml, respectively). The cells are harvested by centrifugation (200×g for 5 minutes) and washed three times in cold RPMI 1640. Approximately 2.5×10⁶ cells are used per 96 well microtitre plate.

Preparation of Spleen Cell Suspension

The mouse is killed by an overdose of anesthetic (Forene), the spleen dissected and pressed through a cell strainer (70 μm mesh cell strainer; Becton & Dickinson, Oxford, UK, Cat. No 2350). The cell suspension is washed three times in RPMI 1640 (as above) and counted: 5.10⁶ cells /96 well plate are necessary.

Fusion of Myeloma Cells and Spleen Cells

The spleen and myeloma cells are mixed (2:1), centrifuged (200×g for 5 min) and the pellet warmed in a 37° C. water bath. Prewarmed polyethylene glycol 4000 (1 ml per 10⁸ cells) is added slowly over one minute, then 20 ml of prewarmed wash medium over two minutes. After centrifugation the pellet is carefully resuspended in selection medium (RPMI 1640, 10% FCS, 1% penicillin-streptomycin, 10% BM condimed H1 (feeder cell replacement from Boehringer Mannheim, Lewes, UK; Cat. No. 1 088 947), 10% HAT-media supplement (hypoxanthine, aminopterin and thymidine to select against unfused myeloma cells; Boehringer Mannheim, Lewes, UK; Cat. No. 644 579) and plated, 200 μl/well of a 96 well microtitre plate.

After five days clusters of hybrid cells can be identified by examining the bottom of the microtitre wells with an inverted microscope. After 10-14 days the culture supernatant is tested for the presence of antibodies by ELISA (Example 5). The positive clones are expanded in a 24 well assay plate and retested.

Cloning of Positive Hybridomas

The expanded clones which are still positive are cloned by limiting dilution. Cells are diluted serially in four dilutions steps in a 96 well microtitre plate; 5, 2, 1 and 0.5 cells/well. HAT-media supplement is replaced with HT-media supplement (Boehringer Mannheim, Lewes, UK; Cat. No. 623 091). After approximately one week the cells are screened by ELISA (Example 5). The cells of those wells containing a single positive clone are expanded.

Production of Monoclonal Antibody Supernatant

The cells are grown in culture flasks in standard medium (RPMI 1640, 10% (v/v) FCS and 1% penicillin-streptomycin) until the hybridomas overgrow and die. The debris is removed by centrifugation and the supernatant containing the antibodies is titred using ELISA (Example 5) before storing under sterile conditions at 4° C., −20° C. or −70° C.

What is claimed is:

1. A pharmaceutical composition comprising as active ingredient (A) a polypeptide comprising the amino acid sequence of SEQ ID NO:2 or SEQ ID NO:16 or a functionally equivalent variant of said amino acid sequence; or (B) a polynucleotide comprising a nucleotide sequence encoding the polypeptide (A); or (C) an antibody which is immunoreactive with the polypeptide (A); or (D) an antisense oligonucleotide comprising a nucleotide sequence complementary to that of polynucleotide (B); optionally together with a pharmaceutically acceptable carrier or diluent.

2. A composition according to claim 1, in which the active ingredient is the polypeptide (A) comprising a portion having at least SO contiguous amino acids from SEQ ID NO:2 or SEQ ID NO:16.

3. A composition according to claim 1, in which the active ingredient is the polynucleotide (B) which is cDNA comprising the nucleotide sequence of SEQ ID NO:1 or SEQ ID NO:15, or a DNA comprising a nucleotide sequence which hybridises to SEQ ID NO:1 or SEQ ID NO:15 under stringent conditions.

4. A composition according to claim 1, in which the active ingredient is the polynucleotide (B) comprising a portion having at least 100 contiguous bases from SEQ ID NO:1 or SEQ ID NO:15.

5. A composition according to claim 1, in which the active ingredient is the polynucleotide (B) comprising a nucleotide sequence encoding at least 50 contiguous amino acids from SEQ ID NO:2 or SEQ ID NO:16.

6. An isolated polynucleotide comprising the nucleotide sequence of SEQ ID NO:15.

7. An isolated polypeptide comprising the amino acid sequence of SEQ ID NO:16.

8. An antibody which is immunoreactive with a polypeptide (A) as specified in claim 1.

9. An antisense oligonucleotide comprising a nucleotide sequence complementary to that of a polynucleotide (B) as specified in claim 1.

10. A polynucleotide probe comprising at least 15 contiguous nucleotides of a polynucleotide (B) as specified in claim 1, or a complement thereof, labelled to provide a detectable signal.

11. A method of treating an inflammatory disease which comprises administering to a subject in need thereof an effective amount of a polypeptide (A) as specified in claim 1, or a polynucleotide (B) as specified in claim 1, or an antibody (C) as specified in claim 1, or an antisense oligonucleotide (D) as specified in claim 1.

12. A method according to claim 11, in which the disease is an inflammatory or obstructive airways disease.

13. A method according to claim 12, in which the disease is asthma or chronic obstructive pulmonary disease.

14. A method of detecting the presence of a polynucleotide (B) as specified in claim 1 in a cell or tissue which comprises contacting DNA from the cell or tissue with a polynucleotide probe comprising at least 1S contiguous nucleotides of a polynucleotide (B) or a complement thereof under conditions where the probe is specifically hybridizable with a polynucleotide (B), and detecting whether hybridization occurs.

15. A method of determining whether a subject has an inflammatory disease, comprising determining, in a cell sample from the subject, the level of expression of a polynucleotide (B) as specified in claim 1, and comparing said level with the level of expression of the polynucleotide in a cell sample from a healthy subject.

16. A method of monitoring treatment of a subject having an inflammatory disease with a drug, which comprises determining the level of expression of a polynucleotide (B) as specified in claim 1, or a polypeptide (A) as specified in claim 1, or the level of an activity of said polypeptide, in a cell sample from the subject following the treatment and comparing said level with the respective level before the treatment.

17. A pair of oligonucleotides useful as primers for amplification of a fragment of a polynucleotide (B) as specified in claim 1, each oligonucleotide of said pair being at least 15 nucleotides in length and said pair having sequences such that when used in a polymerase chain reaction with human genomic DNA or a suitable human cDNA target, they result in synthesis of a DNA fragment containing part or all of the nucleotide sequence of said polynucleotide (B).

18. A method of identifying a substance suitable for use in the treatment of an inflammatory disease comprising combining a candidate-substance with a polypeptide (A) as specified in claim 1 and measuring the effect of the candidate substance on the activity of said polypeptide (A).

19. A method of identifying a substance suitable for use in the treatment of an inflammatory disease which binds to a polypeptide (A) as specified in claim 1 comprising mixing a candidate substance with said polypeptide (A) and determining whether binding has occurred.
